(19) United States
(12) Patent Application Publication
(10) Pub. No.: US 2023/0124053 A1
(43) Pub. Date: Apr. 20, 2023

(54) ELECTRONIC DEVICE FOR MANAGING BEDSORES BASED ON ARTIFICIAL INTELLIGENCE MODEL AND OPERATING METHOD THEREOF

(71) Applicant: SeoHong Tech. Co., Ulsan (KR)

(72) Inventor: Hyun Deok KANG, Seoul (KR)

(73) Assignee: SeoHong Tech. Co., Ulsan (KR)

(21) Appl. No.: 18/047,798

(22) Filed: Oct. 19, 2022

(30) Foreign Application Priority Data
Oct. 19, 2021 (KR) 10-2021-0139390

(51) Int. Cl. A61B 5/00; G16H 50/50; A61G 7/057

(52) U.S. Cl. CPC A61B 5/447; G16H 50/50; A61B 5/6892; A61B 5/743; A61B 5/7267; A61B 5/746; A61G 7/05769; A61B 5/4836

(57) ABSTRACT

Provided are a method for managing bedsores based on an artificial intelligence model and an electronic device performing the same. According to an exemplary embodiment, a method for managing, by an electronic device, bedsores based on an artificial intelligence model may include: acquiring user information of a user who uses a smart mat connected to the electronic device; acquiring at least one type of sensor data from the smart mat connected to the electronic device; when at least one type of sensor data is input, acquiring the bedsores management contents for the user from the artificial intelligence model by inputting the sensor data into the artificial intelligence model that outputs the bedsores management contents for each user according to the user information; and outputting the acquired bedsores management contents.

### CROSS-REFERENCE TO RELATED APPLICATIONS

This application claims the priority of Korean Patent Application No. 10-2021-0139390 filed on Oct. 19, 2021, in the Korean Intellectual Property Office, the disclosure of which is incorporated herein by reference.

### BACKGROUND OF THE INVENTION

### Field of the Invention

The present disclosure relates to a method for managing bedsores based on artificial intelligence technology and an electronic device performing the same. More particularly, the present disclosure relates to an electronic device outputting personal customized bedsores management contents by interlocking with a smart mat based on an artificial intelligence model.

### Description of the Related Art

According to the National Statistical Office, in Korea, it is expected that the aged over 65 years old reach 14.9% in 2019, 20.3% in 2025, and 46.5% in 2067 due to rapid aging. In addition, the increase in demand for medical services due to aging and the burden of medical expenses is expected to increase, and in the future, the medical service paradigm is expected to change to prediction, prevention and personalized center.

The paradigm of the medical service is changing from receiving treatment after the disease, and the center of personality is being changed from personal health, and a smart healthcare industry is emerging as the most important means in medical system innovation having high efficiency, which can cope with the lack of manpower for a healthcare service in the age of aging. On the other hand, technical attempts to organize vast medical data generated by the IoT technology to the healthcare industry are being studied through artificial intelligence technology, and there are also multiple attempts for medical staffs to introduce the technical attempts to the field.

However, in an environment where the elderly population grows rapidly, the level of manpower in the nursing facility is not corresponding to it, making it difficult to manage the patient. In particular, bedsores that require continuous management of medical staff or caregivers are a common disease which may occur for everybody, and even though all patients who are admitted to the hospital as well as nursing hospitals are subject to monitoring, a lot of manpower is required to manage bedsores, so there is a limit to the lack of manpower in the case of bedsores using general mats.

Therefore, in order to help medical staff and caregivers manage efficient patients and solve the lack of manpower, the development of personalized bedsores management technology is required based on artificial intelligence technology.

### SUMMARY OF THE INVENTION

An exemplary embodiment has been made in an effort to provide a method for managing bedsores based on an artificial intelligence model and an electronic device performing the same.

Further, an exemplary embodiment has been made in an effort to provide a method for providing bedsores management contents by an electronic device by interlocking with a smart mat.

According to an exemplary embodiment of the present disclosure, there is provided a method for managing, by an electronic device, bedsores based on an artificial intelligence model, which may include: acquiring user information of a user who uses a smart mat connected to the electronic device; acquiring at least one type of sensor data from the smart mat connected to the electronic device; when at least one type of sensor data is input, acquiring the bedsores management contents for the user from the artificial intelligence model by inputting the sensor data into the artificial intelligence model that outputs the bedsores management contents for each user according to the user information; and outputting the acquired bedsores management contents.

According to another exemplary embodiment of the present disclosure, there is provided an electronic device managing bedsores based on an artificial intelligence model, which may include: a network interface; a memory storing one or more instructions; and at least one processor executing one or more instructions, in which at least one processor executes one or more instructions to acquire user information of a user who uses a smart mat connected to the electronic device; acquire at least one type of sensor data from the smart mat connected to the electronic device; when at least one type of sensor data is input, acquire the bedsores management contents for the user from the artificial intelligence model by inputting the sensor data into the artificial intelligence model that outputs the bedsores management contents for each user according to the user information; and output the acquired bedsores management contents.

According to yet another exemplary embodiment of the present disclosure, there is provided a computer-readable recording medium having a program allowing a computer to execute a method for managing, by an electronic device, bedsores based on an artificial intelligence model, in which the method may include: acquiring use information of a user who uses a smart mat connected to the electronic device; acquiring at least one type of sensor data from the smart mat connected to the electronic device; when at least one type of sensor data is input, acquiring the bedsores management contents for the user from the artificial intelligence model by inputting the sensor data into the artificial intelligence model that outputs the bedsores management contents for each user according to the user information; and outputting the acquired bedsores management contents.

### BRIEF DESCRIPTION OF THE DRAWINGS

The above and other aspects, features and other advantages of the present invention will be more clearly understood from the following detailed description taken in conjunction with the accompanying drawings, in which:

FIG. 1 is a diagram schematically illustrating a process of performing a method for managing bedsores based on an artificial intelligence model by an electronic device according to an exemplary embodiment;

FIG. 2 is a diagram schematically illustrating a process of performing a method for managing bedsores based on an artificial intelligence model by interlocking with a smart mat by the electronic device according to an exemplary embodiment;

FIG. 3 is a diagram schematically illustrating a process of performing a method for training a bedsores prevention model by using an artificial intelligence algorithm and managing bedsores by using the trained bedsores prevention model by the electronic device according to an exemplary embodiment;

FIG. 4 is a flowchart of a method for managing bedsores based on an artificial intelligence model by the electronic device according to an exemplary embodiment;

FIG. 5 is a diagram for specifically describing an example of outputting bedsores management contents by interlocking with the smart mat by the electronic device according to an exemplary embodiment;

FIG. 6 is a diagram for describing a process of generating a feature map in which sensor data values acquired from the smart mat are visually displayed by the electronic device according to an exemplary embodiment;

FIG. 7 is a diagram for describing an example of the feature map generated by the electronic device according to an exemplary embodiment;

FIG. 8 is a diagram for describing a structure of the smart mat according to an exemplary embodiment;

FIG. 9 is a diagram for describing a structure of a pressure sensor in the smart mat according to an exemplary embodiment;

FIG. 10 is a diagram for describing a configuration of an array sensor controller according to an exemplary embodiment;

FIG. 11 is a diagram for describing a process of outputting the bedsores management contents by using the artificial intelligence model by the electronic device according to an exemplary embodiment;

FIG. 12 is a block diagram of an electronic device according to an exemplary embodiment;

FIG. 13 is a block diagram of an electronic device according to another exemplary embodiment; and

FIG. 14 is a block diagram of a server according to another exemplary embodiment.

### DETAILED DESCRIPTION OF THE PREFERRED EMBODIMENT

Terms used in the present specification will be described in brief and the present disclosure will be described in detail.

Terms used in the present disclosure adopt general terms which are currently widely used as possible by considering functions in the present disclosure, but the terms may be changed depending on an intention of those skilled in the art, a precedent, emergence of new technology, etc. Further, in a specific case, a term which an applicant arbitrarily selects is present and in this case, a meaning of the term will be disclosed in detail in a corresponding description part of the invention. Accordingly, a term used in the present disclosure should be defined based on not just a name of the term but a meaning of the term and contents throughout the present disclosure.

Further, throughout the specification, unless explicitly described to the contrary, the word “comprise” and variations such as “comprises” or “comprising”, will be understood to imply the inclusion of stated elements but not the exclusion of any other elements. In addition, terms including “part’, “module”, and the like disclosed in the specification mean a unit that processes at least one function or operation and this may be implemented by hardware or software or a combination of hardware and software.

An exemplary embodiment of the present disclosure will be described more fully hereinafter with reference to the accompanying drawings so as to be easily implemented by those skilled in the art. However, the present disclosure can be realized in various different forms, and is not limited to the exemplary embodiments described herein. In addition, in the drawings, in order to clearly describe the present disclosure, a part not related to the description is not omitted and like reference numerals designate like elements throughout the specification.

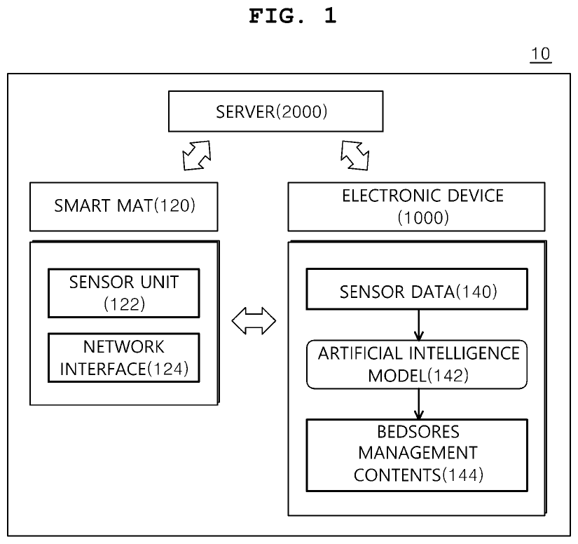

FIG. 1 is a diagram schematically illustrating a process of performing a method for managing bedsores based on an artificial intelligence model by an electronic device according to an exemplary embodiment.

According to an exemplary embodiment, a bedsores management system 10 may include an electronic device 1000, a server 2000, and a smart mat 120. However, according to another exemplary embodiment, the bedsores management system 10 may further include a user terminal (not illustrated) used by a nurse or a doctor. Further, according to an exemplary embodiment, the bedsores management system 10 may also include only the electronic device 1000 and the smart mat 120, of course.

According to an exemplary embodiment, the electronic device 1000 analyzes at least one type of sensor data acquired from the smart sensor by using the artificial intelligence model to provide bedsores management contents for a user who uses the smart mat. For example, when at least one type of sensor data acquired by acquired by a sensor unit 122 of the smart mat 120 is transmitted to the electronic device 1000 through a network interface, the electronic device 1000 acquires sensor data 140 and analyzes the acquired sensor data 140 by using an artificial intelligence model 142 to determine bedsores management contents 144.

According to an exemplary embodiment, the electronic device 1000 simultaneously collects heterogeneous sensor data acquired by the smart mat 120 periodically in real time, and collects, analyzes, and evaluates the collected sensor data based on the artificial intelligence model to provide alarm contents for notifying that a change of a body position is required, body position change schedule contents for a body position change schedule, and bedsores management contents including other bedsores and a health management comprehensive solution for managing a health of a patient.

According to an exemplary, the electronic device 1000 generates a Braden Scale protocol based bedsores patient distinguishing model, and analyzes the sensor data acquired by the smart mat to identify minor and major bedsores patients. Further, the electronic device 1000 interlocks with an application installed in a user terminal used by a guardian, the nurse, or the doctor to transmit the bedsores management contents to the user terminal used by the guardian, the nurse, or the doctor in real time, and help a caretaker to easily manage the patient through information transmitted in real time. Further, according to an exemplary embodiment, the electronic device 1000 may also transmit a control signal for controlling an operation of the smart mat to the smart mat in order to change the body position of the patient based on the bedsores management contents.

According to an exemplary embodiment, the artificial intelligence model used by the electronic device 1000 may be a model which may be learned based on the artificial intelligence learning algorithm. According to an exemplary embodiment, the artificial intelligence model used by the electronic device 1000 may be a model which may include a neural network model. For example, the neural network model as an artificial neural network may be referred to as a computing system focused on a biological neural network. The artificial neural network may learn performing a task by considering multiple samples unlike a classic algorithm that performs the task according to a predefined condition.

According to an exemplary embodiment, the artificial neural network may have a structure in which artificial neurons are connected, and the connection between the neurons may be referred to as a synapse. The neural may process a received signal, and the processed signal may be transmitted to another neuron through the synapse. An output of the neuron may be referred to as activation, and the neuron and/or the synapse may have a weight which may be varied, and an influence of the signal processed by the neuron may increase or decrease.

For example, the neural network model may include layers and a plurality of blocks defined as weights regarding connection intensities of the layers. More specifically, in the neural network model, each of a plurality of neural network layers in the neural network model has a plurality of weight values (or weights), and performs a neural network arithmetic operation through an arithmetic operation result of a previous layer and an arithmetic operation between the plurality of weights. The plurality of weights which the plurality of neural network layers has may be optimized by a learning result of the artificial neural network.

For example, the plurality of weights may be modified and updated so that a loss value or a cost value acquired by the artificial intelligence model (e.g., neural network model) is reduced or minimized during a learning process. The artificial intelligence model used by the electrode device according to the present disclosure may be include a deep neural network (DNN), and is, for example, a Convolutional Neural Network (CNN), a Deep Neural Network (DNN), a Recurrent Neural Network (RNN), a Restricted Boltzmann Machine (RBM), a Deep Belief Network (DBN), a Bidirectional Recurrent Deep Neural Network (BRDNN), a Long Short-Term Memory (LSTM) model, or Deep Q-Networks, but is not limited to the above-described example.

According to an exemplary embodiment, the electronic device 1000 may be a smartphone, a PC, a cellular phone, a laptop, a medial player, a server, or other mobile or non-mobile computing devices, but is not limited thereto. Further, according to an exemplary embodiment, the smart mat 120 may be a structure a mat frame and sensors fastened to a part of the mat frame and acquiring at least one type of sensor data may be fastened, and a device which transmits the acquired sensor data to the electronic device. The smart mat may include heterogeneous sensors, and transmit the heterogeneous sensors to the electronic device at a predetermined period. According to an exemplary embodiment, the sensor unit 122 may include at least one of a temperature sensor or a pressure sensor. However, the present disclosure is not limited thereto, and the sensor unit 122 may include at least one of a humidity sensor, a near infrared sensor, a short-range sensor, and an acceleration sensor.

Further, according to an exemplary embodiment, the electronic device 1000 may be connected to a server 2000. According to an exemplary embodiment, the electronic device 1000 interlocks with the server 2000 to analyze the body position of the user using the smart mat from the sensor data acquired from the smart mat, determine friction force and response force at which the electronic device 1000 contacts a body part of the user, and output bedsores management contents suitable for a current state of the user. Further, according to an exemplary embodiment, the electronic device 1000 may further acquire lifelog information for the user of the smart mat from the server 2000 or other external devices, and further reflects user information determined based on the lifelog information to further provide user-customized bedsores management contents.

According to an exemplary embodiment, the server 2000 may include other computing devices which are connected to the electronic device 1000 through the network to transmit/receive data to/from the electronic device 1000. According to an exemplary embodiment, the server 2000 may be connected to the electronic device 1000 through at least combination of a Local Area Network (LAN), a Wide Area Network (WAN), a Value Added Network (VAN), and a mobile radio communication network. Further, according to an exemplary embodiment, the server 2000 may be a data communication network of a comprehensive meaning, which enables each network constituent subjects (e.g., the electronic device and the server) illustrated in FIG. 1 to smoothly communicate with each other, and may also include a wired Internet, a wireless Internet, and a mobile wireless communication network itself.

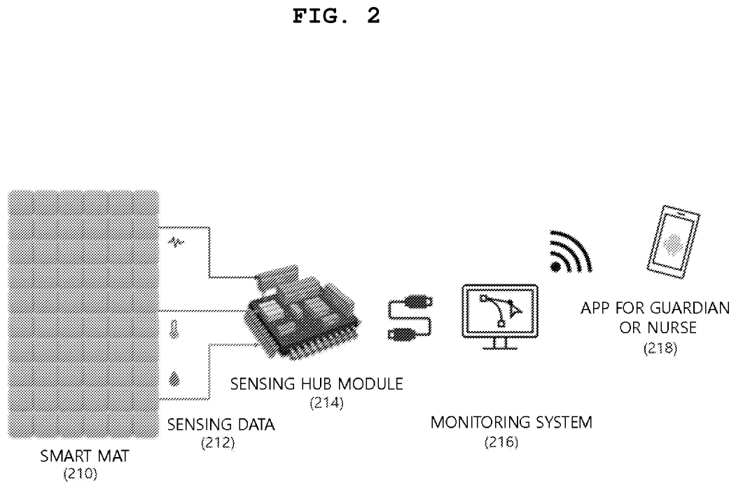

FIG. 2 is a diagram schematically illustrating a process of performing a method for managing bedsores based on an artificial intelligence model by interlocking with a smart mat by an electronic device according to an exemplary embodiment.

According to an exemplary embodiment, the bedsores management system 10 may acquire a plurality of types of sensor data 212 from a smart mat 210. For example, the smart mat 210 may include a sensor hub module 214, and transmit heterogeneous types of sensor data to a monitoring system 216 through the sensor hub module 214. According to an exemplary embodiment, the monitoring system 216 may be a component included in the electronic device 1000 or the server 2000.

The monitoring system 216 analyzes the plurality of sensor data acquired from the sensor hub module based on the artificial intelligence module to determine the bedsores management contents and transmits the determined bedsores management contents to a user terminal for the guardian or the nurse to allow the guardian or the nurse to view the bedsores management contents in real time.

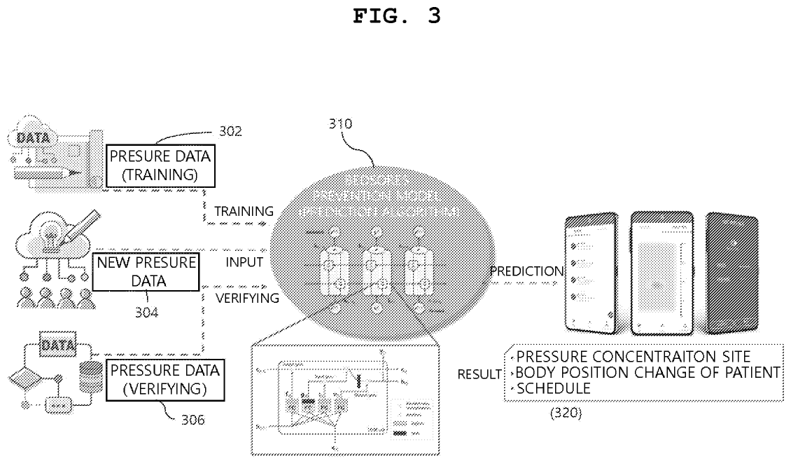

FIG. 3 is a diagram schematically illustrating a process of performing a method for training a bedsores prevention model by using an artificial intelligence algorithm and managing bedsores by using the trained bedsores prevention model by the electronic device according to an exemplary embodiment.

According to an exemplary embodiment, the electronic device 1000 may acquire training pressure data 320, and train an artificial intelligence model 310 based on the acquired pressure data. According to an exemplary embodiment, the artificial intelligence model 310 may be a bedsores prevention model performing a prediction algorithm. The electronic device 1000 may provide bedsores management contents including pressure concentration site information, patient's body position change information, or schedule information by inputting new pressure data 304 into the trained artificial intelligence model 310.

Further, according to an exemplary embodiment, the electronic device 1000 may set some of the acquired pressure data as verifying pressure data 306, and verify at least one sensor data itself or an output result of the artificial intelligence model 310 based on the verification pressure data. The electronic device 1000 applies the artificial intelligence model to pressure data newly acquired from a smart mat used by a target patient to output the bedsores management contents only when a verification result shows a reliability of a predetermined threshold valid value or more.

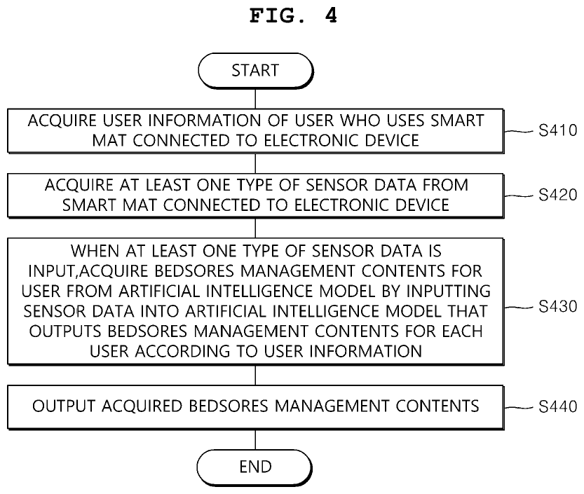

FIG. 4 is a flowchart of a method for managing bedsores based on an artificial intelligence model by an electronic device according to an exemplary embodiment.

In S310, the electronic device 1000 may acquire user information of the user who uses the smart mat connected to the electronic device. For example, the electronic device 1000 may also acquire the user information based on a user input for the electronic device, and also acquire identification information for the smart mat and acquire user information matching the identification information, and also acquire each of the identification information and the user information, and also matching and store the acquired identification information and user information.

According to an exemplary embodiment, the user information may include Internet use history information of the user, SNS information, lifelong information, name information, age information, and medial history information, but is not limited thereto.

In S420, the electronic device 1000 may acquire at least one type of sensor data from the smart mat connected to the electronic device. According an exemplary embodiment, the electronic device 1000 may acquire at least one type of sensor data from the smart mat connected to the electronic device at a predetermined period. According an exemplary embodiment, the electronic device 1000 may acquire at least one sensor data of pressure data, humidity data, or temperature data. Further, according an exemplary embodiment, the electronic device 1000 may acquire at least one of the pressure data, the temperature data, or the humidity data.

Although not illustrated in FIG. 4, acquiring at least one type of sensor data by the electronic device may include acquiring identification data of at least one type of sensors disposed in the smart mat and acquiring positional data of at least one type of sensors disposed in the smart mat. For example, the electronic device 1000 may further acquire the positional data of the sensor data and identification data capable of uniquely identifying the sensor data in acquiring the sensor data from at least one type of sensors in the smart mat.

Further, although not illustrated in FIG. 4, the electronic device 1000 may verify at least one type of acquired sensor data after acquiring at least one type of sensor data. According to an exemplary embodiment, the electronic device 1000 may acquire the bedsores management contents by inputting the sensor data into the artificial intelligence model when a validity of at least one type of sensor data is identified to be equal to or more than a predetermined threshold valid value based on a result of verifying at least one type of sensor data.

According to an exemplary embodiment, the electronic device 1000 may also transmit the sensor data to the server connected to the electronic device when the validity of at least one type of sensor data is identified to be equal to or more than the threshold valid value based on the result of verifying at least one type of sensor data. Further, according an exemplary embodiment, at least one type of sensor data acquired from the smart sensor by the electronic device 1000 may include at least one of the pressure data, the humidity data, or the temperature data. Therefore, the smart mat may include at least one of the humidity sensor, the temperature sensor, or the pressure sensor.

In step S430, when at least one type of sensor data is input, the electronic device 1000 may acquire the bedsores management contents for the user from the artificial intelligence model by inputting the sensor data into the artificial intelligence model that outputs the bedsores management contents for each user according to the user information. According to an exemplary embodiment, the electronic device 1000 may generate a feature map in which a change of sensor data values according to the user's body position is displayed by using at least one type of sensor data, and acquire the bedsores management contents by inputting the feature map into the artificial intelligence model.

According to an exemplary embodiment, although not illustrated in FIG. 4, the electronic device 1000 may generate a feature map including color regions displayed with different colors according to sizes of values of sensor data at locations according to positional data based on the positional data of at least one type of sensor data after acquiring at least one type of sensor data. The electronic device 1000 may also acquire the bedsores management contents from the artificial intelligence model by inputting at least one of at least one type of sensor data or the feature map into the artificial intelligence model.

In S440, the electronic device 1000 may output the bedsores management contents acquired from the artificial intelligence model. According to an exemplary embodiment, the bedsores management contents output by the electronic device 1000 may include at least one of patient's body type information, alarm contents for notifying occurrence of an error for a predetermined region according to the patient's body type information, and body position change schedule contents regarding a body position change schedule. According to an exemplary embodiment, the bedsores management contents may include temperature information, friction information, response force information, and time information contacting the mat for reach body site region of the patient identified according to the patient's body type information.

Further, although not illustrated in FIG. 4, according to another exemplary embodiment, the electronic device 1000 may also transmit the bedsores management contents to another electronic device or server connected to the electronic device. For example, the electronic device 1000 may transmit the bedsore management contents to the user terminal used by the nurse, the guardian, or the doctor in real time.

Further, although not illustrated in FIG. 4, acquiring the bedsores management contents by the electronic device 1000 may further include a series of following operations. For example, the electronic device 1000 may identify a user's body type map image indicating information on a user body type based on the user information and the positional data of at least one type of sensor data. The electronic device 1000 may identify an image region indicating an abnormal friction force state or an image region indicating an abnormal contamination state in the user's body type map image. The electronic device 1000 may generate body position change schedule contents for the identified body site region and acquire bedsores management contents including the generated body position change schedule contents.

Further, although not illustrated in FIG. 4, the electronic device 1000 may acquire at least one type of sensor data from the smart mat again after outputting the bedsores management contents, and reacquire the bedsores management contents by inputting at least one type of sensor data which is acquired again into the artificial intelligence model. The electronic device 1000 may also train the artificial intelligence model based on a comparison result of the reacquired bedsores management contents and the previously acquired bedsores management contents.

Further, the electronic device 1000 may also transmit, to the smart mat, a control signal for controlling air pressure of an air pocket in the smart mat corresponding to an image region indicating the abnormal friction force state or an image region indicating the abnormal contamination state after outputting the bedsores management contents. For example, the electronic device 1000 identifies the user's body site indicating a specific abnormal state based on the bedsores management content and transmits a control signal for controlling the air pocket of the smart mat corresponding to the identified user's body site to the smart mat to prevent the abnormal body site from contacting the surface of the smart mat for a predetermined threshold time or more.

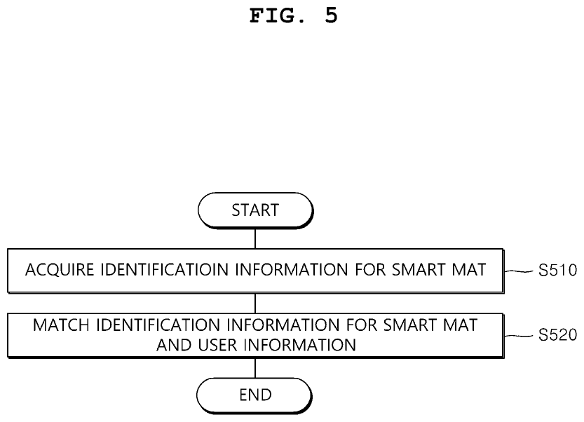

FIG. 5 is a diagram for specifically describing an example of outputting bedsores management contents by interlocking with the smart mat by the electronic device according to an exemplary embodiment.

In S510, the electronic device 1000 may acquire identification information for the smart mat. For example, unique identification information capable of identifying each smart mat device may be allocated to the smart mat, and the electronic device 1000 may acquire the identification information while acquiring the sensor data acquired from the smart mat or acquire the identification information from the smart mat before acquiring the sensor data from the smart mat. According to an exemplary embodiment, the electronic device 1000 may also acquire the user information including the identification information.

In S520, the electronic device 1000 may match the identification information for the smart mat and the user information. For example, the electronic device 1000 matches and stores unique identification information acquired from the smart mat and the user information of the user acquired by the electronic device to match and store data for a specific user and sensor data acquired from a specific smart mat.

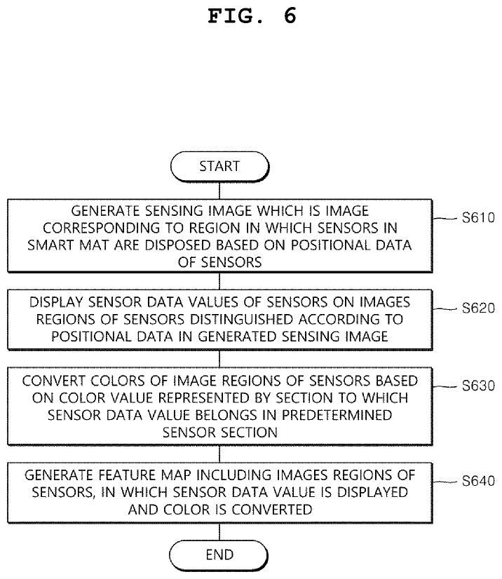

FIG. 6 is a diagram for describing a process of generating a feature map in which sensor data values acquired from the smart mat are visually displayed by the electronic device according to an exemplary embodiment.

In S610, the electronic device 1000 may generate a sensing image which is an image corresponding to a region where sensors are disposed in the smart mat based on positional data of the sensors. For example, the electronic device 1000 may acquire both the sensor data and positional data of the sensor data where the sensor data are acquired from at least one type of sensor data in the smart mat. The electronic device 1000 may generate the sensing image by imaging a region where the sensors in the smart mat are disposed based on the positional data.

In S620, the electronic device 1000 may display sensor data values of the sensors on the image regions of sensors distinguished according to the positional data in the sensing image. The electronic device 1000 may distinguish the image regions of the sensors on the sensing image. The image regions of the respective sensors may correspond to locations of the sensors in the smart mat. The electronic device 1000 may determine the image regions of the sensors at the locations corresponding to the locations of the sensors in the smart mat based on the positional data on the sensing image, and display the sensor data values in the determined image regions of the image regions. According to an exemplary embodiment, the sensor data value may be displayed by symbols such as characters and numbers, but also schematized and expressed as images, or colors, or pictures.

In S630, the electronic device 1000 may convert the colors of the image regions of the sensors based on a color value represented by a section to which the sensor data value in a predetermined sensor section belongs. For example, the electronic device 1000 may previously set a data section to which the sensor data value acquired from each sensor belongs. The respective data sections may match predetermined color sections. The electronic device 1000 may identify the color matching the data section to which the sensor data of each sensor belongs, and convert the color of the image region of each sensor based on the identified color.

In step S640, the electronic device 1000 may generate a feature map displaying the sensor data value, and including the images regions of the sensors in which the colors are converted. For example, the electronic device 1000 identifies the image region of each sensor according to the positional data on the sensing image and converts the color of the image region of each sensor based on each sensor data value of each sensor data to generate the feature map. The feature map generated by the electronic device 1000 may include the positional data of the sensors, the sensor data, and the sensor image region converted into the color value of the section to which the sensor data value belongs.

Further, although not illustrated in FIG. 6, the electronic device 1000 may also determine the friction force for each of the image regions of the sensors distinguished according to each positional data based the sensor data value after generating the feature map. According to an exemplary embodiment, the electronic device 1000 may also determine the response force for each of the image regions of the sensors distinguished according to the positional data based on the sensor data value.

For example, the electronic device 1000 may acquire a plurality of sensor data based on a predetermined period, and then generate a feature map according to a predetermined frame interval. The electronic device 1000 may generate the user's body type map information based on a distribution pattern of the sensor data acquired on the feature map, and track a predetermined sensor image region on a feature map corresponding to a next frame based on the user's body type map information. The electronic device 1000 may identify the corresponding sensor image region as a region in a state of friction force or response force or more when all sensor data values shown on the tracked predetermined sensor image region are identified to be a predetermined data value or more within a predetermined threshold time on the feature map.

According to another exemplary embodiment, the electronic device 1000 may also identify an abnormal region more accurately by reflecting an influence according to toss and turning of the patient. For example, the electronic device 1000 may identify the corresponding sensor image region as the region in the state of friction force or response force or more when a movement distance of the specific sensor image region tracked on the feature map is equal to or more than a predetermined threshold distance and all sensor data values in a specific sensor image which moves a threshold distance or more are identified to be a predetermined data value or more within the predetermined threshold time on the feature map.

The electronic device 1000 may provide bedsores management contents including the alarm contents or the schedule contents for the patient's body site corresponding to the corresponding sensor image region.

The electronic device 1000 may also generate a feature map to which data for the friction force and the response force are reflected in addition to the pressure data, the temperature data, or the humidity data by reflecting the data for the friction force and the response force to the generated feature map.

The electronic device 1000 may also acquire the bedsores management contents from the artificial intelligence model by inputting at least one type of sensor data or the feature map to which the data for the friction force and the response force are reflected into the artificial intelligence model.

Further, according to an exemplary embodiment, the electronic device 1000 may identify the image region showing the state of the friction force or the response force or more in the feature map by using the feature map for the friction force and the response force by using the artificial intelligence model. Further, according to an exemplary embodiment, the electronic device 1000 may also identify an image region showing the abnormal contamination state due to sweat or urine in the feature map based on the sensor data value after identifying the mage region showing the state of the friction or response force or more.

Further, when at least one of the image region showing the state of the friction force or more or the image region showing the abnormal contamination state is identified, the electronic device 1000 may acquire alarm contents for notifying occurrence of abnormality for the corresponding region as the bedsores management contents.

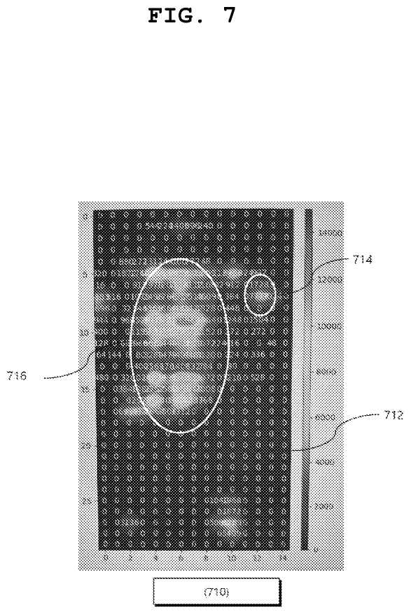

FIG. 7 is a diagram for describing an example of the feature map generated by the electronic device according to an exemplary embodiment.

According to an exemplary embodiment, a feature map 710 may be expressed as a matrix 712 having a size corresponding to the number of sensors in the smart mat. According to an exemplary embodiment, the feature map 710 may include the positional data indicating the locations of the sensors in the smart mat, the sensor data value at the location according to the positional data, and the images regions of the sensors displayed with the color according to the data section to which the sensor data value belongs. According to an exemplary embodiment, a color of a sensor image region may be converted in which a sensor data value 714 according to specific positional data is displayed and the corresponding data value is displayed based on a color to which a color to which the sensor data value 714 belongs in the feature map.

According to an exemplary embodiment, the electronic device 1000 may identify the corresponding sensor image region as a very high pressure state and display the corresponding sensor image region with a red color when a pressure data value belongs to a first data section, identify the corresponding sensor image region as a high pressure state and display the corresponding sensor image region with a yellow color when the pressure data value belongs to a second data section, identify the corresponding sensor image region as an intermediate pressure state and display the corresponding sensor image region with a green color when the pressure data value belongs to a third data section, and identify the corresponding sensor image region as a low pressure state and display the corresponding sensor image region with a blue color when the pressure data value belongs to a fourth data section. However, the present disclosure is not limited to the above-described example, and the electronic device 1000 may generate the feature map by displaying image regions of sensors with different colors according to a predetermined data section.

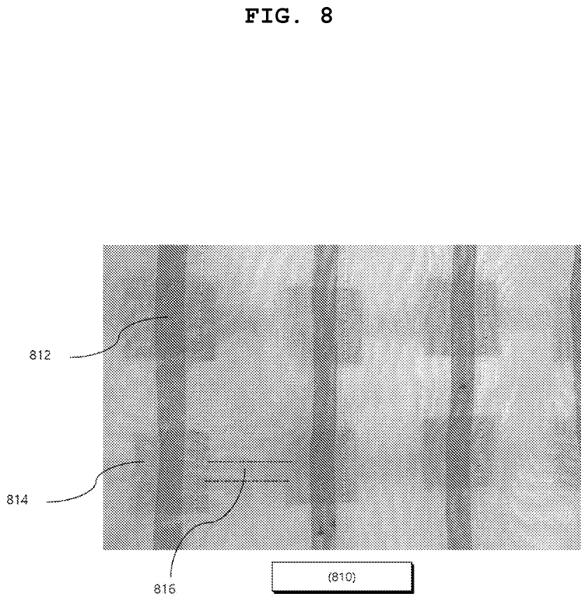

FIG. 8 is a diagram for describing a structure of the smart mat according to an exemplary embodiment.

According to an exemplary embodiment, the smart mat may include at least one type of sensor 814, a first pattern line 812 connecting sensors to a vertical axis, and a second pattern line 816 connecting the sensors to a horizontal axis. Further, although not illustrated in FIG. 8, the smart mat 810 may further include a processor for controlling at least one type of sensor, a memory storing one or more instructions for defining the operation of the processor, a network interface for transmitting sensor data to the electronic device, and a sensor hub module managing each sensor data.

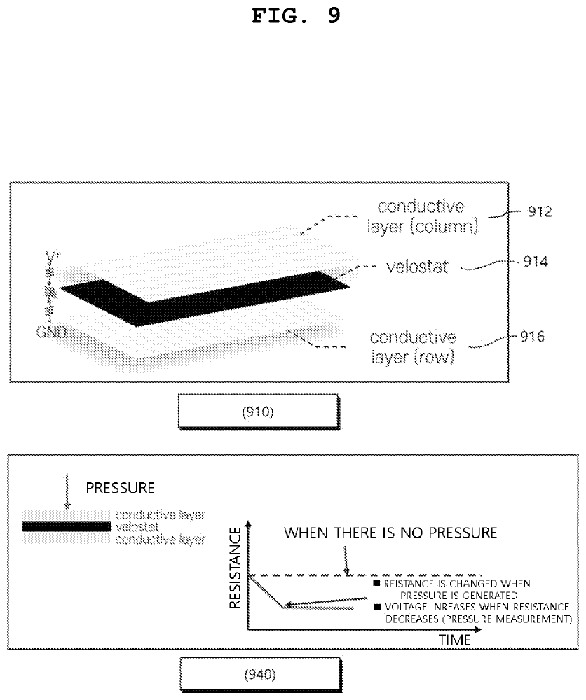

FIG. 9 is a diagram for describing a structure of a pressure sensor in the smart mat according to an exemplary embodiment.

Referring to a FIG. 910 of FIG. 9, the structure of the pressure sensor in the smart matter is illustrated. According to an exemplary embodiment, the pressure sensor may include a first conductive layer 912, a velostat 914, and a second conductive layer 916. According to an exemplary embodiment, the first conductive layer 912 and the second conductive layer 916 may constitute the pattern line illustrated in FIG. 8.

Referring to a FIG. 940, when a pressure depending on a body weight of the patient is applied, a change amount of a resistance value of a unit circuit provided as the pressure sensor is illustrated. The pressure sensor may sense a resistance value change depending on a pressure change amount when the pressure depending on the body weight of the patient is applied, and acquires voltage data depending on the resistance value change as pressure data. The processor of the smart mat may determine a pressure value based on a voltage data value, and control the network interface to transmit the pressure value to the electronic device.

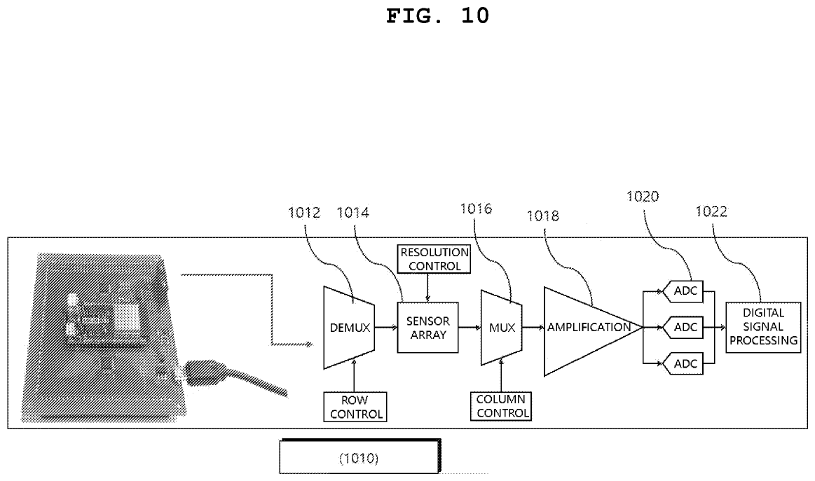

FIG. 10 is a diagram for describing a configuration of an array sensor controller according to an exemplary embodiment.

Referring to a FIG. 1010, an array sensor controller for controlling sensors disposed in an array form in the smart mat is illustrated. According to an exemplary embodiment, the array sensor controller may include a demultiplexer 1012, a sensor array 1014, a multiplexer 1016, an amplifier 1018, an analog digital converter 1020, and a digital signal processor 1022. Since those skilled in the art may intuitively infer respective components in the array sensor controller from names thereof, a detailed description will be omitted.

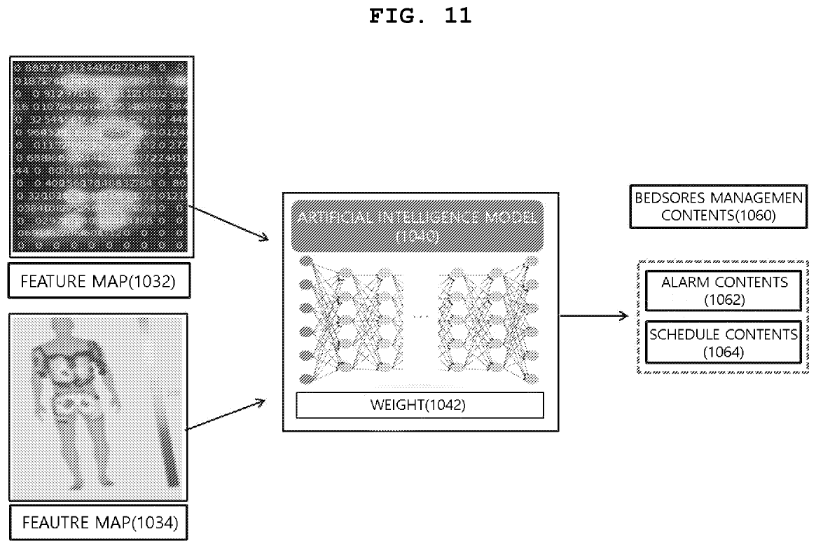

FIG. 11 is a diagram for describing a process of outputting the bedsores management contents by using the artificial intelligence model by the electronic device according to an exemplary embodiment.

According to an exemplary embodiment, the electronic device 1000 may acquire at least one type of sensor data from the smart mat and generate feature maps 1032 and 1034 based on at least one type of acquired sensor data. According to an exemplary embodiment, the electronic device 1000 may acquire bedsores management contents 1060 from the artificial intelligence model by inputting at least one of at least one type of sensor data or the feature map into an artificial intelligence model 1040.

Further, according to an exemplary embodiment, the electronic device 1000 acquires training sensor data and trains the artificial intelligence model 1040 based the acquired training sensor data to pretrain the artificial intelligence model to output predetermined bedsores management contents 1060 depending on the sensor data. According to an exemplary embodiment, a process of training the artificial intelligence mode by the electronic device 1000 may correspond to a process of modifying and updating weights for connection strengths of predetermined layers and nodes in the artificial intelligence model.

According to an exemplary embodiment, the bedsores management contents 1060 may include alarm contents 1062 and schedule contents 1064. However, the present disclosure is not limited the above-described example, and the bedsores management contents 1060 may further include body type information of a smart mat user, friction force and response force information for each body site indicated by the body type information, and contact time information for a time when each body site continuously contacts the mat, of course.

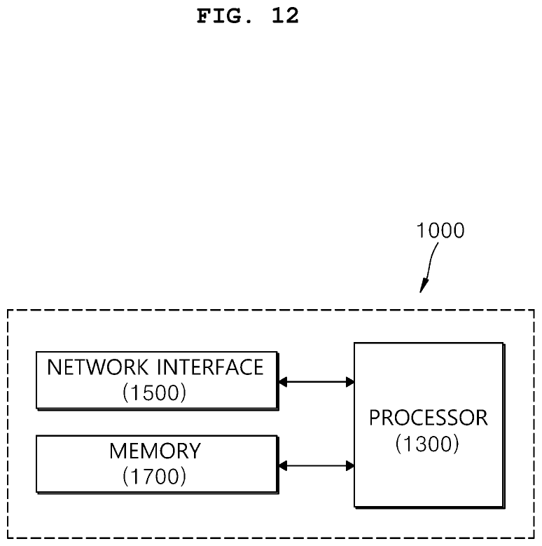

FIG. 12 is a block diagram of an electronic device according to an exemplary embodiment.

FIG. 12 is a block diagram of an electronic device according to another exemplary embodiment.

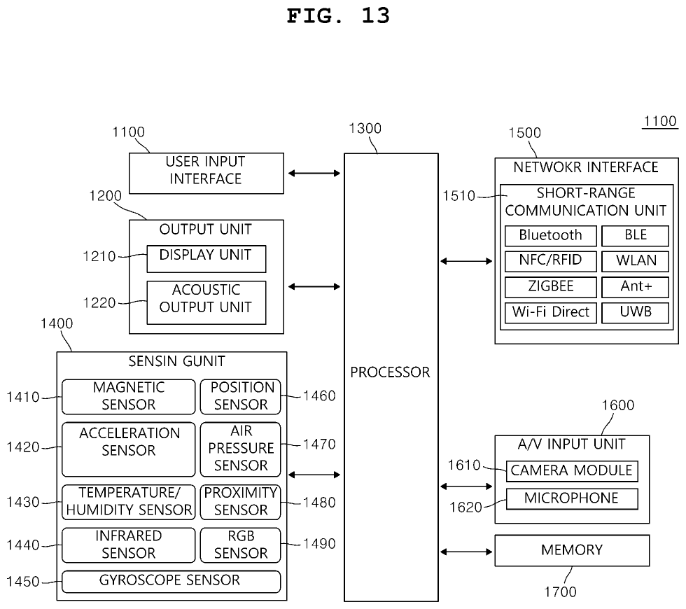

According to an exemplary embodiment, the electronic device 1000 may include a processor 1300, a network interface 1500, and a memory 1700. However, not all illustrated components are required. The electronic device 1000 may be implemented by more components than the illustrated components and the electronic device 1000 may be implemented by fewer components. For example, as illustrated in FIG. 13, the electronic device 1000 according to an exemplary embodiment may further include a user input interface 1100, an output unit 1200, a sensing unit 1400, and an A/V input unit 1600 in addition to the processor 1300, the network interface 1500, and the memory 1700.

The user input interface 1100 means a means used for a user to input a sequence for controlling the electronic device 1000. For example, the user input interface 1100 may include a key pad, a dome switch, a touch pad (a contact type capacitance scheme, a pressure type resistance film scheme, an infrared sensing scheme, a surface ultrasound conduction scheme, an integral tension measurement scheme, a piezo effect scheme, etc.) a jog wheel, a jog switch, etc., but is not limited thereto. The user input interface 1100 may receive an input sequence of the user for a screen which the electronic device 1000 outputs on a display. Further, the user input interface 1100 may also receive a touch input of the user who touches the display or a key input through a graphic user interface on the display.

The output unit 1200 which is used for outputting an audio signal or a video signal may include a display unit 1210 and an acoustic output unit 1220. For example, the display unit 1210 may display the feature map, the sensor data, and the bedsores management contents, the alarm contents, and the schedule contents determined based on the sensor data.

The acoustic output unit 1220 outputs audio data received from the network interface 1500 or stored in the memory 1700. Further, the acoustic output unit 1220 outputs an acoustic signal related to a function performed by the electronic device 1000. The acoustic output unit 1220 may include a speaker, a buzzer, and the like. Further, according to an exemplary embodiment, the acoustic output unit 1220 may acoustically output the alarm content depending on an increase in bedsores occurrence possibility for a specific body site of the smart mat user identified by the electronic device 1000. For example, the acoustic output unit may output a notice sound or a warning sound when it is determined that the bedsores occurrence possibility for the specific body site is high based on the bedsores management contents output from the artificial intelligence model.

The sensing unit 1400 may include at least one of a magnetic sensor 1410, an acceleration sensor 1420, a temperature/humidity sensor 1430, an infrared sensor 1440, a gyroscope sensor 1450, a position sensor (e.g., GPS) 1460, an air pressure sensor 1470, a proximity sensor 1480, and an RGB sensor (illuminance sensor) 1490, but is not limited thereto. Since those skilled in the art may intuitively infer functions of the respective sensors from names thereof, a detailed description will be omitted.

The processor 1300 generally controls all operations of the electronic device 1000. For example, the processor 1300 executes programs stored in the memory 1700 to wholly control the user input interface 1100, the output unit 1200, the sensing unit 1400, the communication unit 1500, the A/V input unit 1600, and the memory 1700.

According to an exemplary embodiment, the processor 1300 may acquire the user information of the user information of the user who uses the smart mat connected to the electronic device and acquire at least one type of sensor data from the smart mat connected to the electronic device, and when at least one type of sensor data is input, the processor 1300 inputs the sensor data into the artificial intelligence model that outputs the bedsores management contents for each user depending on the user information to acquire the bedsores management contents for the user from the artificial intelligence model and output the acquired bedsores management contents.

According to an exemplary embodiment, the processor 1300 may transmit the bedsores management contents to another electronic device connected to the electronic device.

According to an exemplary embodiment, the electronic device 1300 verifies at least one type of sensor data after acquiring at least one type of sensor data, and when the validity of at least one type of sensor data is identified to be equal to or more than a predetermined threshold valid value based on the verification result, the processor 1300 inputs the sensor data into the artificial intelligence model to acquire the bedsores management contents.

According to an exemplary embodiment, the electronic device 1300 may transmit the sensor data to the server connected to the electronic device when the validity of at least one type of sensor data is identified to be equal to or more than the threshold valid value based on the result of verifying at least one type of sensor data.

According to an exemplary embodiment, the processor 1300 may acquire identification information for the smart mat and match the identification information for the smart mat and the user information.

According to an exemplary embodiment, the processor 1300 may include at least one of pressure data, humidity data, or temperature data from the smart mat.

According to an exemplary embodiment, the processor 1300 may acquire identification data of at least one type of sensor disposed in the smart mat, and acquire positional data of at least one type of sensor disposed in the smart mat.

According to an exemplary embodiment, the processor 1300 generates a feature map including color regions displayed with different colors according to sizes of values of the sensor data at a location depending on the positional data based on the positional data of at least one type of sensor data after acquiring at least one type of sensor data, and inputs at least one type of sensor data and the feature map into the artificial intelligence model to acquire the bedsores management contents from the artificial intelligence model.

According to an exemplary embodiment, the processor 1300 may generate a sensing image which is an image corresponding to a region where the sensors in the smart mat are disposed based on the positional data of the sensors, display sensor data values of the sensors in image regions of sensors distinguished according to the positional data in the generated sensing image, convert colors of the image regions of the sensors based on a color value shown by a section to which the sensor data value belongs within a predetermined sensor section, and generate a feature map including the image regions of the sensors in which the sensor data values are displayed and the colors are converted.

According to an exemplary embodiment, after generating the feature map, the processor 1300 determines the friction force for each of the image regions of the sensors distinguished according to the positional data based on the sensor data value, determines the response force for each of the image regions of the sensors distinguished according to the positional data based on the sensor data value, reflects the data for determined friction force and response force to the generated feature map, and inputs at least one type of sensor data, and the feature map to which the data for the friction force and the response force are reflected into the artificial intelligence model to acquire the bedsores management contents from the artificial intelligence model.

According to an exemplary embodiment, the processor 1300 may identify the image region indicating the abnormal friction force state in the feature map by using the feature map for the friction force and the response force, identify the image region indicating the abnormal contamination state due to the sweat or urine in the feature map based on the sensor data value, and when at least one of the image region indicating the abnormal friction force state or the image region indicating the abnormal contamination state is identified, the processor 1300 may acquire alarm contents for notifying the occurrence of the abnormality for the corresponding region as the bedsores management contents.

According to an exemplary embodiment, the processor 1300 may identify the user's body type map image based on the user information and the positional data of at least one type of sensor data, identify a body site region corresponding to the image region indicating the abnormal friction force state or the image region indicating the abnormal contamination state in the user's body type map image, and generate body position change schedule contents for the identified body site region.

According to an exemplary embodiment, the processor 1300 may acquired the generated body position change schedule contents as the bedsores management contents.

According to an exemplary embodiment, after outputting the bedsores management contents, the processor 1300 acquires at least one type of sensor data from the smart mat again and inputs at least one type of sensor data which is acquired again into the artificial intelligence model to required the bedsores management contents, and train the artificial intelligence model based on a comparison result of the reacquired bedsores management contents and the acquired bedsores management contents.

According to an exemplary embodiment, the processor 1300 may also transmit a control signal for controlling air pressure of an air pocket in the smart mat corresponding to an image region indicating the abnormal friction force state or an image region indicating the abnormal contamination state after outputting the bedsores management contents.

According to an exemplary embodiment, the electronic device 1000 may include one or more components which allow the electronic device 1000 to communicate with another device (not illustrated) and the server 2000. Another device (not illustrated) may be a computing device such as the electronic device 1000 or a sensing device, but is not limited thereto. For example, the network interface 1500 may include a short-range communication unit 1510 or a long-range communication unit (not illustrated).

The short-range wireless communication unit 1510 may include a Bluetooth communication unit, a Bluetooth low energy (BLE) communication unit, a hear filed communication unit, a WLAN (WiFi) communication unit, a Zigbee communication unit, an infrared data association (IrDA) communication unit, a Wi-Fi direct (WFD) communication unit, an ultra wideband (UWB) communication unit, an Ant+ communication unit, etc., but is not limited thereto.

The long-range communication unit may include a mobile communication unit or a broadcast receiving unit. For example, the mobile communication module transmits/receives at least one radio signal to at least one of a base station, an external terminal, and a server on a mobile communication network. Here, the radio signal may include various types of data depending on transmitting/receiving a voice signal, a video communication call signal, or a text/multimedia message. The broadcast receiving unit receives a broadcasting signal and/or broadcasting related information from the outside through a broadcasting channel. The broadcasting channel may include a satellite channel and a terrestrial channel. According to an implementation example, the electronic device 1000 may not include a broadcast receiving unit (not illustrated), of course.

According an exemplary embodiment, the network interface 1500 may acquire at least one type of sensor data from the smart mat connected to the electronic device 1000. Further, the network interface 1500 may also acquire lifelog information of the user from the server 2000 connected to the electronic device. Further, the network interface 1500 may also acquire both sensor data and positional data at which the sensor data are acquired from the smart sensor, of course.

The audio/video (A/V) input unit 1600 which is used for inputting an audio signal or a video signal may include a camera 1610 and a microphone 1620. The camera 1610 may obtain an image frame such as a still image or a moving picture for shoes or a shoes wearer through an image sensor in a video communication mode or a photographing mode. An image captured through the image sensor may be processed through the processor 1300 or a separate image processing unit (not illustrated).

The microphone 1620 may receive the acoustic signal from the external device or the user. The microphone 1620 may receive a voice input of the user. The microphone 1620 may use various noise removal algorithms for removing noise generated in the process of receiving an external acoustic signal.

The memory 1700 may store a program for processing and controlling the processor 1300, and also store data input into the electronic device 1000 or output from the electronic device 1000. Further, the memory 1700 may also store information on the artificial intelligence model used by the electronic device 1000. According to an exemplary embodiment, the memory 1700 may further store training data information used for training the artificial intelligence model by using the electronic device 1000, and also further store parameter information for the artificial intelligence model.

For example, when models based on neural networks which are already generated are modified in addition to the trained neural network, the memory 1700 may further store layers of the modified models, and information on a weight between the layers.

The memory 1700 may include at least one type of storage medium of a flash memory type storage medium, a hard disk type storage medium, a multimedia card micro type storage medium, a card type memory (for example, an SD or XD memory, or the like), a random access memory (RAM), a static random access memory (SRAM), a read-only memory (ROM), an electrically erasable programmable read-only memory (EEPROM), a programmable read-only memory (PROM), a magnetic memory, a magnetic disk, and an optical disk.

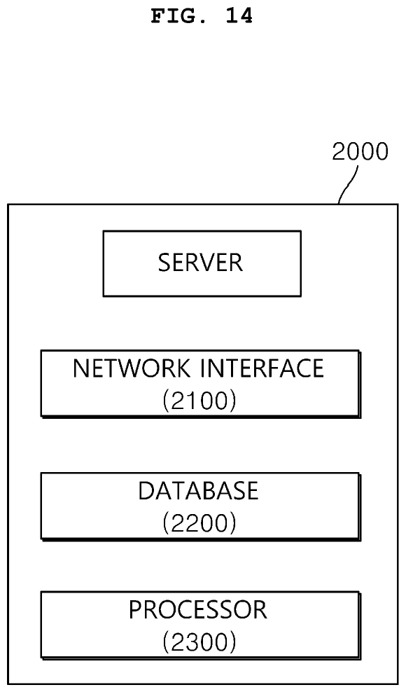

FIG. 14 is a block diagram of a server according to another exemplary embodiment.

The server 2000 may include a network interface 2100, a database 2200, and a processor 2300. The network interface 2100 may correspond to the network interface 1500 of the electronic device 1000 illustrated in FIGS. 12 and 13. For example, the network interface 2100 may acquire at least one type of sensor data, bedsores management contents, and information on the feature map from the electronic device 1000. Further, the network interface 2100 may also transmit user lifelog information to the server.

The processor 2300 generally controls all operations of the server 2000. For example, the processor 2300 executes programs stored in a DB 2200 of the server 2000 to generate the DB 2200 and the network interface 2100 as a whole. Further, the processor 2300 executes the programs stored in the DB 2100 to perform some or all of the operations of the electronic device 1000 in FIGS. 1 to 11.

For example, the processor 2300 acquires at least one type of sensor data from the smart mat and analyzes at least one type of sensor data by using the artificial intelligence model to autonomously determine the bedsores management contents and transmit the determined bedsores management contents to the electronic device, of course.

Further, according to an exemplary embodiment, the processor 2300 may acquire training sensor data acquired from the electronic device or the smart mat, and also autonomously train the artificial intelligence model based on the acquired training sensor data. The processor 2300 may also transmit the layers in the trained artificial intelligence model and weight information regarding a connection strength of the layers to the electronic device.

The database 2200 may correspond to the memory 1700 of the electronic device 1000 illustrated in FIG. 12. For example, the database 2200 may also store training data received from the electronic device 1000, and information (e.g., neural network model layers and weight values regarding the connection strength of the layers) on the artificial intelligence model trained based on the training data.

The method according to an exemplary embodiment may be implemented in a form of a program command which may be performed through various computer means and recorded in the computer readable medium. The computer readable medium may include a program command, a data file, a data structure, etc., singly or combinationally. The program command recorded in the medium may be specially designed and configured for the present disclosure, or may be publicly known to and used by those skilled in the computer software field.

Further, a computer program device may be provided, which includes a recording medium storing a program to perform the method according to an exemplary embodiment. An example of the computer readable recording medium includes magnetic media, such as a hard disk, a floppy disk, and a magnetic tape, optical media such as a CD-ROM and a DVD, magneto-optical media such as a floptical disk, and hardware devices such as a ROM, a RAM, and a flash memory, which are specially configured to store and execute the program command. An example of the program command includes a high-level language code executable by a computer by using an interpreter and the like, as well as a machine language code created by a compiler. While the exemplary embodiment of the present disclosure has been described, it is to be understood that the present disclosure is not limited to the disclosed exemplary embodiments, but, on the contrary, is intended to cover various modifications and equivalent arrangements included within the spirit and scope of the appended claims.

What is claimed is:

1. A method for managing, by an electronic device, bedsores based on an artificial intelligence model, the method comprising:
acquiring user information of a user who uses a smart mat connected to the electronic device;
acquiring at least one type of sensor data from the smart mat connected to the electronic device;
when at least one type of sensor data is input, acquiring the bedsores management contents for the user from the artificial intelligence model by inputting the sensor data into the artificial intelligence model that outputs the bedsores management contents for each user according to the user information; and
outputting the acquired bedsores management contents.

2. The method of claim 1, wherein the method includes transmitting the bedsores management contents to another electronic device connected to the electronic device.

3. The method of claim 1, wherein the method includes after the acquiring of at least one type of sensor data,
verifying at least one type of sensor data; and
acquiring the bedsores management contents by inputting the sensor data into the artificial intelligence model when a validity of at least one type of sensor data is identified to be equal to or more than a predetermined threshold valid value based on the verification result.

4. The method of claim 3, wherein the method further includes transmitting the sensor data to a server connected to the electronic device when the validity of at least one type of sensor data is identified to be equal to or more than the threshold valid value based on the result of verifying at least one type of sensor data.

5. The method of claim 3, wherein the acquiring of the user information further includes
acquiring identification information for the smart mat, and
matching the identification information for the smart mat and the user information.

6. The method of claim 3, wherein the acquiring of at least one type of sensor data includes acquiring at least one of pressure data, humidity data, or temperature data from the smart mat.

7. The method of claim 6, wherein the acquiring of at least one type of sensor data further includes
acquiring identification data of at least one type of sensor disposed in the smart mat, and
acquiring positional data of at least one type of sensor disposed in the smart mat.

8. The method of claim 7, wherein the method further includes generating a feature map including color regions displayed with different colors according to sizes of values of the sensor data at a location depending on the positional data based on the positional data of at least one type of sensor data after the acquiring of at least one type of sensor data, and
the acquiring of the bedsores management contents includes
acquiring the bedsores management contents from the artificial intelligence model by inputting at least one type of sensor data and the feature map into the artificial intelligence model.

9. The method of claim 8, wherein the generating of the feature map includes
generating a sensing image which is an image corresponding to a region in which sensors in the smart mat are disposed based on the positional data,
displaying the sensor data values of the sensors on images regions of sensors distinguished according to the positional data in the generated sensing image,
converting colors of the image regions of the sensors based on a color value represented by a section to which the sensor data value belongs in a predetermined sensor section, and
generating a feature map including the images regions of the sensors, in which the sensor data value is displayed and the color is converted.

10. The method of claim 9, wherein the method includes
determining friction force for each of the image regions of the sensors distinguished according to the positional data based on the sensor data value after the generating of the feature map,
determining response force for each of the image regions of the sensors distinguished according to the positional data based on the sensor data value, and
reflecting data for the determined friction force and the response force onto the generated feature map, and
the acquiring of the bedsores management contents includes
acquiring the bedsores management contents from the artificial intelligence model by inputting the feature map to which the data for the friction force and the response force is reflected into the artificial intelligence model.

11. The method of claim 10, wherein the acquiring of the bedsores management contents includes
identifying an image region showing an abnormal friction force state in the feature map by using the feature map for the friction force and the response force,
identifying an image region showing an abnormal contamination state due to sweat or urine in the feature map based on the sensor data value, and
when at least one of the image region showing the state of the friction force or more or the image region showing the abnormal contamination state is identified, acquiring alarm contents for notifying occurrence of abnormality for the corresponding region as the bedsores management contents.

12. The method of claim 10, wherein the acquiring of the bedsores management contents includes
identifying user's body type map image based on the user information and the positional data of at least one type of sensor data,
identifying a body site region corresponding to the image region showing the abnormal friction force state or an image region indicating an abnormal contamination state in the user's body type map image in the user's body type map image,
generating body position change schedule contents for the identified body site region, and
acquiring the generated body position change schedule contents as the bedsores management contents.

13. The method of claim 12, wherein the method includes
acquiring at least one type of sensor data from the smart mat again after outputting the bedsores management contents,
reacquiring the bedsores management contents by inputting at least one type of sensor data which is acquired again into the artificial intelligence model, and
training the artificial intelligence model based on a comparison result of the reacquired bedsores management contents and the acquired bedsores management contents.

14. The method of claim 12, wherein the method further includes
after the outputting of the bedsores management contents,
transmitting a control signal for controlling an air pressure in an air pocket in the smart mat corresponding to the image region showing the abnormal friction force state or an image region indicating an abnormal contamination state in the user's body type map image.

15. An electronic device managing bedsores based on an artificial intelligence model, comprising:
a network interface;
a memory storing one or more instructions; and
at least one processor executing one or more instructions,
wherein at least one processor executes one or more instructions to
acquire user information of a user who uses a smart mat connected to the electronic device;
acquire at least one type of sensor data from the smart mat connected to the electronic device;
when at least one type of sensor data is input, acquire the bedsores management contents for the user from the artificial intelligence model by inputting the sensor data into the artificial intelligence model that outputs the bedsores management contents for each user according to the user information; and
output the acquired bedsores management contents.

16. A computer-readable recording medium having a program allowing a computer to execute a method for managing, by an electronic device, bedsores based on an artificial intelligence model, where the method includes:
acquiring use information of a user who uses a smart mat connected to the electronic device;
acquiring at least one type of sensor data from the smart mat connected to the electronic device;
when at least one type of sensor data is input, acquiring the bedsores management contents for the user from the artificial intelligence model by inputting the sensor data into the artificial intelligence model that outputs the bedsores management contents for each user according to the user information; and
outputting the acquired bedsores management contents.
